(19) United States
(12) Patent Application Publication
(10) Pub. No.: US 2025/0387405 A1
(43) Pub. Date: Dec. 25, 2025

(54) PHARMACEUTICAL COMPOSITION CONTAINING PLATINUM DRUGS OR PLATINUM DRUG COCRYSTALS, AND USE THEREOF

(71) Applicant: MEDONCARE PHARMACEUTICAL CO., LTD, Changsha, Hunan (CN)

(72) Inventors: Xiaozhong LIU, Changsha, Hunan (CN); Qifeng JIN, Changsha, Hunan (CN); Chuxiong LI, Changsha, Hunan (CN); Xiong CAI, Changsha, Hunan (CN); Feng ZHANG, Changsha, Hunan (CN)

(21) Appl. No.: 18/713,580

(22) PCT Filed: Nov. 25, 2022
(86) PCT No.: PCT/CN2022/134287

§ 371 Date: May 24, 2024

(30) Foreign Application Priority Data
Nov. 26, 2021 (CN) 202111427808.4

(51) Int. Cl. A61K 31/555; A61K 45/06; A61P 19/02

(52) U.S. Cl. CPC A61K 31/555; A61K 45/06; A61P 19/02

(57) ABSTRACT

A pharmaceutical use of a pharmaceutical composition containing platinum drugs or platinum drug co-crystals as main active substances in preventing or treating immunological diseases such as rheumatoid arthritis, and other diseases. The platinum drugs mainly refer to oxaliplatin, and the platinum drug co-crystals mainly refer to carboplatin co-crystals or oxaliplatin co-crystals. Further disclosed is a method using platinum drugs or platinum drug co-crystals, either alone or in combination with at least one additional therapeutic agent or adjuvant therapy agent.

### FIELD OF THE INVENTION

The invention relates to a pharmaceutical preparation containing a platinum drug or a platinum-based pharmaceutical cocrystal (platinum drug co-crystal) as the main active substance and a preparation method thereof, and the use for preventing or treating autoimmune diseases such as rheumatoid arthritis and other diseases. A method of using the platinum-based drug or platinum-based cocrystal alone or in combination with at least one other therapeutic reagent or adjuvant is also disclosed.

### BACKGROUND OF THE INVENTION

Rheumatoid arthritis (RA) is a chronic, systemic autoimmune disease that erodes joint synovium and cartilage. Its clinical manifestations are swelling and pain of joint, inflammatory injury of cartilage, narrowing of joint space, and even joint stiffness, deformity and movement dysfunction (Best Pract Res Clin Rheumatol, 2018, 32 (2): 174-187).

As a chronic inflammatory disease, RA has complex pathogenic factors, involving heredity, immune regulation and external infection, and no clear conclusion has been reached in clinical studies. Although there have been breakthroughs in the treatment of rheumatoid arthritis, there have been no effective drugs or treatment methods for the radical cure of rheumatoid arthritis. The development of drugs for the treatment of rheumatoid arthritis has been a research hotspot in the field of autoimmune diseases (Arthritis Research & Therapy, 2019, 21(1): 949-958).

Combined with the guidelines for the treatment of Rheumatoid Arthritis in 2021 issued by the American Rheumatic Society (ACR), the antirheumatic drugs (DMARDs) currently used to improve the condition of RA mainly include traditional synthetic DMARDs (such as hydroxychloroquine, sulfasalazine, methotrexate, leflunomide), biological DMARDs (TNF inhibitors such as adalimumab, T cell inhibitors such as abaxil, IL-6 receptor inhibitors such as topirazumab, anti-CD20 antibodies such as rituximab), and Targeted synthesis of DMARDs (JAK inhibitors such as topatib, baritinib, etc.) and glucocorticoids (such as cortisone, dexamethasone, prednisone, etc.). In addition, the study of natural drugs against rheumatoid arthritis, such as sinomenine, Tripterygium wilfordii, Radix Paeoniae Alba, Jintiesuo, aconitine, etc. has also attracted widespread attention (Research on the mechanism of natural drugs against rheumatoid arthritis based on nuclear transcription factor κB signaling pathway, China Pharmacy, 2019, 30(7): 1004-1008).

Although different types of DMARDs have outstanding anti-inflammatory and immunosuppressive effects, the selectivity for different conditions is limited, and the long-term use of these drugs is easy to produce large adverse reactions, and the resistance is also an important reason. Although natural drugs have achieved certain efficacy in the treatment of rheumatoid arthritis, its mechanism and material basis need to be further studied.

### SUMMARY OF THE INVENTION

The invention relates to the use of pharmaceutical preparations containing oxaliplatin or oxaliplatin cocrystal or carboplatin cocrystal as main active substances in the prevention or treatment of autoimmune diseases such as rheumatoid arthritis and other diseases.

The invention provides a method of using oxaliplatin or oxaliplatin cocrystal or carboplatin cocrystal alone or in combination with at least one other therapeutic reagent or adjuvant therapeutic agent for the treatment of RA. The method of using oxaliplatin or oxaliplatin cocrystal or carboplatin cocrystal of the invention can meet one or more objectives in preventing or treating RA disease, such as but not limited to low dosage, long period of administration interval, significant control of RA disease progression and recurrence, etc.

On the one hand, the platinum metal-containing complexes including oxaliplatin are commonly used in the first-line treatment of tumors in clinical practice. Platinum-based drugs with different structures correspond to their different structure-activity relationships, and their anticancer efficacy and toxicity are greatly affected by their structures. Platinum-based drugs with different structures have different solubility and stability, and the mechanism of action belongs to the alkylation mechanism. However, the adaptability of platinum-based drugs with different structures to different tumors also varies. (Research progress in modern structure of platinum complexes. Eur J Med Chem, 2017, 140:349-382). The side effects and resistance of platinum-based drugs limit their therapeutic research in related fields such as anti-tumor. Because of this, platinum-based drugs are no longer considered to be the main focus of new drug development or disease treatment. The potential research directions of platinum-based drugs in the field of anti-tumor are: to look for compounds with better efficacy, lower toxicity and improved pharmaceutical properties than maternal compounds (cisplatin and carboplatin), to expand the anticancer spectrum, and to develop new drugs that have no cross-resistance to cisplatin and carboplatin.

On the other hand, the use of platinum-based drugs in the treatment and research of immune system diseases has rarely been reported. Some studies have found that patients diagnosed with RA develop head and neck squamous cell carcinoma in the later stage of treatment, involving the neck, nasal cavity and tongue. RA was found to be effectively relieved after cisplatin treatment (Omar and Adimulam BMC Musculoskeletal Disorders 2013, 14 Suppl 1: A5), but no further studies or application reports have been reported. The reason may be that cisplatin may cause serious side effects such as nephrotoxicity and ototoxicity during treatment, especially the generation of cisplatin resistance, which becomes the most important factor limiting the clinical efficacy of cisplatin. In addition, in view of the complex factors affecting the pathogenesis of RA, involving genetic and immune regulation and external infection factors, the mechanism of cisplatin as a treatment of RA can't be determined.

As early as 1929, transition metal gold (Au) was used to treat rheumatoid arthritis and achieved results. Gold preparations are polymers formed by gold elements and sulfhydryl compounds or gold sulfates. Gold preparations for clinical application are divided into water-soluble injections and fat-soluble oral agents. Gold preparation has anti-inflammatory effect and immune regulation by inhibiting the phagocytic activity of inflammatory cells and reducing the release of lysosomal enzymes (Critical Reviews in Oncology/Hematology, 2002, 42:225248). Similar to cisplatin, gold preparation has not been widely used in clinic because of its toxic and side effects.

In the process of studying the clinical application of platinum-based compounds and their cocrystal compounds in the treatment of tumors and other indications, we found for the first time the specific effects of different platinum-based compounds and their cocrystal compounds or preparations on RA, and there were significant differences in the efficacy and safety of platinum-based compounds with different structures on RA, and this difference may be the key to determine the value of its clinical application.

In one aspect, we found that dicycloplatin (a hypermeric compound composed of carboplatin and 1,1-cyclobutane dicarboxylic acid by hydrogen bonding) not only has a good effect in the field of prevention or treatment of cancer diseases, but also has a clear effect in the treatment of RA and the treatment of cancer pain (cocrystal composition and its drug use, CN 10753030A). Preliminary studies have shown that dicycloplatin exhibits different pharmacokinetic characteristics from its precursor carboplatin, and the relevant mechanism of action is under further study.

On the other hand, although it belongs to the platinum class of drugs like cisplatin and carboplatin, oxaliplatin is very different from them in terms of efficacy, pharmacokinetics, anti-tumor profile and cytotoxicity, and the most significant is that it does not produce cross-resistance. The central platinum atom of oxaliplatin is surrounded by oxalic acid and 1,2-diaminocyclohexane. This structural difference makes oxaliplatin have different activities compared with other platinum-based drugs, and can activate different cell damage recognition mechanisms. It is possible that oxaliplatin has potential advantages in improving and treating RA because it is not cross-resistant with cisplatin and carboplatin in the treatment of tumors.

On the one hand, the invention provides a pharmaceutical composition comprising one or more of oxaliplatin, oxaliplatin cocrystal and carboplatin cocrystal and a preparation and use method thereof.

On the other hand, one or more of oxaliplatin, oxaliplatin cocrystal and carboplatin cocrystal in the pharmaceutical composition provided by the invention can be the main active pharmaceutical ingredient. In some embodiments, one or more of oxaliplatin, oxaliplatin cocrystal and carboplatin cocrystal may be the only active substance in the pharmaceutical composition. In some embodiments, the pharmaceutical composition contains oxaliplatin. In some embodiments, the pharmaceutical composition contains oxaliplatin cocrystal or carboplatin cocrystal. In some embodiments, the pharmaceutical composition consists of one or more of oxaliplatin, oxaliplatin cocrystal and carboplatin cocrystal; in other embodiments, the pharmaceutical composition consists of an effective dose of one or more of oxaliplatin, oxaliplatin cocrystal and carboplatin cocrystal and at least one additional therapeutic agent or adjuvant therapeutic agent. In some embodiments, oxaliplatin cocrystal may be selected from the group consisting of a cocrystal formed by oxaliplatin and 1,1-cyclobutane dicarboxylic acid, a cocrystal formed by oxaliplatin and chloroquine, and a cocrystal formed by oxaliplatin and polydatin. In some embodiments, carboplatin cocrystal may be selected from the group consisting of a cocrystal formed by carboplatin and chloroquine, and a cocrystal formed by carboplatin and polydatin. In some embodiments, the pharmaceutical composition may further contain at least one therapeutic agent or an adjunct therapeutic agent. In some embodiments, at least one therapeutic agent or an adjunctive therapeutic agent described in the pharmaceutical composition is selected from the group consisting of chloroquine, hydroxychloroquine, methotrexate, curcumin, polydatin, glutathione and aloe vera. In some embodiments, the pharmaceutical composition further contains a pharmaceutically acceptable carrier or excipient. In some embodiments, the pharmaceutical composition may be administered by oral, buccal mucosa, inhalation spray, sublingual, transdermal, transmucosal, topical, muscular, subcutaneous, intradermal, or intravenous route.

In some embodiments, the invention further provides a platinum-based cocrystal, which is a cocrystal formed by oxaliplatin and 1,1-cyclobutane dicarboxylic acid, a cocrystal formed by oxaliplatin and chloroquine, a cocrystal formed by oxaliplatin and polydatin, a cocrystal formed by carboplatin and chloroquine, and a cocrystal formed by carboplatin and polydatin.

In some embodiments, the present invention provides a cocrystal formed by oxaliplatin and 1,1-cyclobutane dicarboxylic acid, preferably the cocrystal formed by oxaliplatin and 1,1-cyclobutane dicarboxylic acid contains one or more of the following diffraction peaks in the XRPD profile: 7.2°±0.2°, 9.3°±0.2°, 10.2°±0.2°, 13.1°±0.2°, 14.3°±0.2°, 15°±0.2°, 19°±0.2°, 21.3°±0.2°, 21.9°±0.2° and 22.6°±0.2°, for example, basically consistent with or consistent with FIG. 1. The invention further provides a cocrystal formed by oxaliplatin and polydatin. Preferably, the cocrystal formed by oxaliplatin and polydatin contains one or more of the following diffraction peaks in the XRPD pattern: 3.4°±0.2°, 6.8°±0.2°, 10°±0.2°, 14.3°±0.2°, 20.4°±0.2°, 22.6°±0.2°, 25.6°±0.2°, and 28.6°±0.2°, for example, basically consistent with or consistent with FIG. 2.

In the present invention, “basically consistent with Figure X” means 90% consistent with Figure X.

The invention relates to a pharmaceutical composition based on one or more of oxaliplatin, oxaliplatin cocrystal and carboplatin cocrystal that provides a sufficient level of bioavailability to maintain a time period level that is therapeutically effective.

On the other hand, the present invention relates to use of a pharmaceutical composition of the present invention wherein an effective amount of the compound is used alone or combined with at least one therapeutic agent or adjuvant therapeutic agent to treat an immune disease. The immune diseases include, but are not limited to, rheumatoid arthritis.

On the other hand, the invention relates to the use of the pharmaceutical composition of the invention in the preparation of medicaments wherein an effective amount of the compound is used alone or combined with at least one therapeutic agent or adjuvant therapeutic agent for the treatment of an immune disease. The immune diseases include, but are not limited to, rheumatoid arthritis.

The pharmaceutical composition according to the invention may be administered by any of the conventional routes, suitable routes may include oral, buccal mucosa, inhaled spray, sublingual, transdermal, transmucosal, local, muscular, subcutaneous, intradermal, or intravenous route or other modes of administration. The preferred mode of administration is determined according to the characteristics of the disease being treated and the specific conditions of the patient.

On the other hand, the present invention relates to methods for the treatment of diseases such as immune diseases such as rheumatoid arthritis, including the administration of the said pharmaceutical composition to the subject, where the pharmaceutical composition has a therapeutic effective amount. In some embodiments, the therapeutic effective amount of the pharmaceutical composition is approximately 0.01 to approximately 10 mg/kg body weight, and in some specific embodiments is approximately 0.01 to approximately 5 mg/kg body weight. The preferred dosage of oxaliplatin or oxaliplatin cocrystal or carboplatin cocrystal depends on the particular disease being treated and the specific circumstances of the patient.

In some embodiments for the treatment of diseases such as immune diseases such as rheumatoid arthritis, the composition combines at least one other therapeutic agent or adjuvant therapeutic agent. At least one of the therapeutic agents or an adjunctive therapeutic agent therein is selected from the group consisting of chloroquine, hydroxychloroquine, methotrexate, curcumin, polydatin, glutathione, and aloe vera. The preferred dosage depends on the particular disease being treated and the specific circumstances of the patient.

In addition, the following technical schemes are provided in the present invention.

In the first aspect, platinum drug cocrystal is provided, which is a cocrystal formed by oxaliplatin and 1,1-cyclobutane dicarboxylic acid, a cocrystal formed by oxaliplatin and chloroquine, a cocrystal formed by oxaliplatin and polydatin, a cocrystal formed by carboplatin and chloroquine, or a cocrystal formed by carboplatin and polydatin.

In some embodiments, the present invention provides a cocrystal formed by oxaliplatin and 1, 1-cyclobutane dicarboxylic acid, preferably a cocrystal formed by oxaliplatin and 1, 1-cyclobutane dicarboxylic acid containing one or more of the following diffraction peaks in the XRPD profile: 7.2°±0.2°, 9.3°±0.2°, 10.2°±0.2°, 13.1°±0.2°, 14.3°±0.2°, 15°±0.2°, 19°±0.2°, 21.3°±0.2°, 21.9°±0.2° and 22.6°±0.2°, for example, basically consistent with or consistent with FIG. 1.

In some embodiments, the cocrystal is a cocrystal formed by oxaliplatin and polydatin, preferably a cocrystal formed by oxaliplatin and polydatin containing one or more of the following diffraction peaks in the XRPD pattern: 3.4°±0.2°, 6.8°±0.2°, 10°±0.2°, 14.3°±0.2°, 20.4°±0.2°, 22.6°±0.2°, 25.6°±0.2°, and 28.6°±0.2°, for example, basically consistent with or consistent with FIG. 2.

In some embodiments, the cocrystal is the cocrystal formed by oxaliplatin and chloroquine. Preferably, the cocrystal formed by oxaliplatin and chloroquine contains one or more of the following diffraction peaks in the XRPD pattern: 4.6°±0.2°, 9.3°±0.2°, 16.4°±0.2°, 19°±0.2°, 20.6°±0.2°, and 21.6°±0.2°, for example, basically consistent with or consistent with FIG. 3.

In some embodiments, the cocrystal is the cocrystal formed by carboplatin and chloroquine. Preferably, the cocrystal formed by carboplatin and chloroquine contains one or more of the following diffraction peaks in the XRPD spectrum: 9.2°±0.2°, 10.2°±0.2°, 11.1°±0.2°, 12.2°±0.2°, 13.4°±0.2°, 14.8°±0.2°, 15.3°±0.2°, 16.6°±0.2°, 20.3°±0.2°, and 20.5°±0.2°. For example, it is basically consistent with or consistent with FIG. 4.

In some embodiments, the cocrystal is the cocrystal formed by carboplatin and polydatin. Preferably, the cocrystal formed by carboplatin and polydatin contains one or more of the following diffraction peaks in the XRPD pattern: 8.6°±0.2°, 12.1°±0.2°, 15.5°±0.2°, 15.8°±0.2°, 17.3°±0.2°, and 20.3°±0.2°, e.g. basically consistent with or consistent with FIG. 5.

In the present invention, “basically consistent with Figure X” means that there is 90% agreement with Figure X.

In the second aspect, a pharmaceutical composition is provided, which contains platinum-based cocrystal, wherein platinum-based cocrystal is selected from oxaliplatin cocrystal or carboplatin cocrystal, wherein oxaliplatin cocrystal is selected from the group consisting of a cocrystal formed by oxaliplatin and 1,1-cyclobutane dicarboxylic acid, a cocrystal formed by oxaliplatin and chloroquine, and a cocrystal formed by oxaliplatin and polydatin; and carboplatin cocrystal is selected from the group consisting of a cocrystal formed by carboplatin and chloroquine, and a cocrystal formed by carboplatin and polydatin.

In some embodiments, the platinum-based cocrystal described in the pharmaceutical composition is defined in the first aspect.

In some embodiments, the platinum-based cocrystal in the pharmaceutical composition is the principal or only active substance of the pharmaceutical composition and, preferably, one or more of oxaliplatin cocrystal and carboplatin cocrystal is the principal or only active substance of the pharmaceutical composition.

In some embodiments, the pharmaceutical composition may further contain at least one therapeutic agent or one adjunctive therapeutic agent.

In some embodiments, at least one therapeutic agent or one adjuvant therapeutic agent described in the pharmaceutical composition is selected from the group consisting of chloroquine, hydroxychloroquine, methotrexate, curcumin, polydatin, glutathione, and aloe vera.

In some embodiments, the pharmaceutical composition further contains a pharmaceutically acceptable carrier or excipient.

In some embodiments, the pharmaceutical composition may be administered by oral, buccal mucosa, inhalation spray, sublingual, transdermal, transmucosal, topical, muscular, subcutaneous, intradermal, or intravenous route.

In the third aspect, a method for the treatment of disease in a subject in need is provided, including the administration to the subject of cocrystal or oxaliplatin in the first aspect or a pharmaceutical composition in the second aspect or a pharmaceutical composition containing oxaliplatin in a therapeutically effective amount.

In some embodiments the disease in question is an autoimmune disease.

In some embodiments, the autoimmune disease in question is rheumatoid arthritis.

In some embodiments, the therapeutic effective amount of the cocrystal or oxaliplatin or pharmaceutical composition is approximately 0.01 to approximately 10 mg/kg body weight.

In some embodiments, the therapeutic effective dose of the cocrystal or oxaliplatin or the pharmaceutical composition is approximately 0.01 to approximately 5 mg/kg body weight.

In some embodiments, the pharmaceutical composition is an aqueous composition, or a suspended composition consisting of at least one therapeutic or adjunctive therapeutic agent dissolved or dispersed in a pharmaceutically acceptable amount.

In the fourth aspect, the use of cocrystal or oxaliplatin in the first aspect or a pharmaceutical composition in the second aspect or a pharmaceutical composition containing oxaliplatin in the preparation of medicaments for the treatment of an autoimmune disease, preferably rheumatoid arthritis, is provided.

### BRIEF DESCRIPTION OF THE DRAWINGS

The apparatus for detecting drug cocrystal structure in the invention is as follows:

X-ray powder diffractometer, manufactured by Shimadzu Company, Japan, model XRD-6000X, Cu—K (α), tube voltage 40 kV, tube current 40 mA, scanning speed 2°/min.

FIG. 1 shows the PXRD pattern of oxaliplatin and 1,1-cyclobutane dicarboxylic acid and their cocrystal, wherein OXA is oxaliplatin; CBDA is 1,1-cyclobutane dicarboxylic acid; OXA-CBDA is oxaliplatin-1,1-cyclobutane dicarboxylic acid cocrystal;

FIG. 2 shows the PXRD patterns of oxaliplatin, polydatin and their cocrystal, wherein OXA is oxaliplatin; PDT is polydatin; OXA-PDT is oxaliplatin-polydatin cocrystal;

FIG. 3 shows the PXRD patterns of oxaliplatin, chloroquine and their cocrystal, wherein OXA is oxaliplatin; U72 is chlorine Quine; OXA-U72 is oxaliplatin-chloroquine cocrystal;

FIG. 4 shows the PXRD patterns of carboplatin and chloroquine and their cocrystal, wherein: CBT is carboplatin; U72 is chloroquine; CBT-U72 is carboplatin-chloroquine cocrystal;

FIG. 5 shows the PXRD patterns of carboplatin and polydatin and their cocrystal, wherein CBT is carboplatin; PDT is polydatin; CBT-PDT is carboplatin-polydatin cocrystal;

FIG. 6 shows the inhibitory effect of oxaliplatin on the proliferation of human fibroblast-like synovial cells in rheumatoid arthritis (HFLS-RA) after treatment with different concentrations of oxaliplatin, and the concentration of oxaliplatin in HFLS-RA is 1.563 μg/mL, 3.125 μg/mL, 6.25 μg/mL, 12.5 μg/mL, 25 μg/mL, 50 μg/mL, 100 μg/mL; IC50=4.56 μg/mL.

FIG. 7 shows the inhibition of MH7A cell proliferation after treatment with different concentrations of oxaliplatin, and the concentration of oxaliplatin in MH7A is 1.563 μg/mL, 3.125 μg/mL, 6.25 μg/mL, 12.5 μg/mL, 25 μg/mL, 50 g/mL, 100 μg/mL; IC50=8.91 μg/mL.

FIG. 8 shows the changes of arthritis index after drug treatment for adjuvant arthritis in rats.

FIG. 9 shows the changes in the degree of joint swelling of the hind foot and ankle after drug treatment for adjuvant arthritis in rats.

### DETAILED DESCRIPTION OF THE INVENTION

The present invention provides a method for preventing, treating rheumatoid arthritis, alleviating or relieving its symptoms and/or slowing or stopping the progression of rheumatoid arthritis, which includes administering effective doses of a pharmaceutical composition containing one or more of oxaliplatin, oxaliplatin cocrystal and carboplatin cocrystal.

In some embodiments, the pharmaceutical composition may contain oxaliplatin. In some embodiments, the pharmaceutical composition may contain oxaliplatin cocrystal. In some embodiments, the pharmaceutical composition may contain carboplatin cocrystal. In some embodiments, the pharmaceutical composition may be composed of one or more of oxaliplatin, oxaliplatin cocrystal and carboplatin cocrystal. In some embodiments, the pharmaceutical composition may contain one or more of oxaliplatin, oxaliplatin cocrystal and carboplatin cocrystal and at least one additional therapeutic agent or adjuvant. Additional therapies or adjuvants can be selected from, but not limited to, hydroxychloroquine, methotrexate, curcumin, polydatin, glutathione, aloe vera or a combination of them. Depending on the characteristics of the disease to be treated, the additional therapeutic agent or adjuvant may contain a known drug. In some embodiments, the additional therapeutic agent or adjuvant may contain and be clinically accepted for the treatment or prevention of the disease.

In some embodiments, the pharmaceutical composition may contain one or more of oxaliplatin, oxaliplatin cocrystal and carboplatin cocrystal and pharmaceutically acceptable carriers or excipients. The term “pharmaceutically acceptable carrier” or “pharmaceutically acceptable excipient” is intended to include any and all solvents, dispersing media, coating agents, antibacterial and antifungal agents, isotonic agents, absorption retardants and inert ingredients. The application of such pharmaceutically acceptable carriers or pharmaceutically acceptable excipients to active pharmaceutical ingredients is well known. In addition, other active pharmaceutical ingredients, such as other drugs, may also be incorporated into said compositions and methods.

In certain embodiments, the pharmaceutical composition of this application may be used for the prevention or treatment of rheumatoid arthritis, and the pharmaceutical composition comprising one or more of oxaliplatin, oxaliplatin cocrystal and carboplatin cocrystal may be administered by oral or injectable routes. In certain embodiments, the pharmaceutical composition of this application may be used to prevent or treat rheumatoid arthritis, which include administering the pharmaceutical compositions of oxaliplatin for at least one, two, or three weeks.

In some embodiments, wherein the pharmaceutical composition of the application may be used for the prevention or treatment of rheumatoid arthritis, the dose range of oxaliplatin or oxaliplatin cocrystal or carboplatin cocrystal in the administered pharmaceutical composition may be approximately 0.01 to approximately 10 mg/kg body weight, approximately 0.01 to approximately 5 mg/kg body weight, approximately 0.01 to approximately 2.5 mg/kg body weight, approximately 0.1 to approximately 10 mg/kg body weight, approximately 0.1 to approximately 5 mg/kg body weight, approximately 0.1 to approximately 2.5 mg/kg body weight, and approximately 0.1 to approximately 1 mg/kg body weight in certain specific embodiments.

### Examples

It can be seen from FIG. 1 that oxaliplatin-1,1-cyclobutane dicarboxylic acid cocrystal (OXA-CBDA) has characteristic diffraction peaks at 7.2°±0.2°, 9.3°±0.2°, 10.2°±0.2°, 13.1°±0.2°, 14.3°±0.2°, 15°±0.2°, 19°±0.2°, 21.3°±0.2°, 21.9°±0.2° and 22.6°±0.2°, which are obviously different from those of raw materials (OXA and CBDA), while the main characteristic peaks in OXA and CBDA disappear in cocrystal compound. It shows that OXA and CBDA form cocrystal compound with new crystal structure by hydrogen bonding.

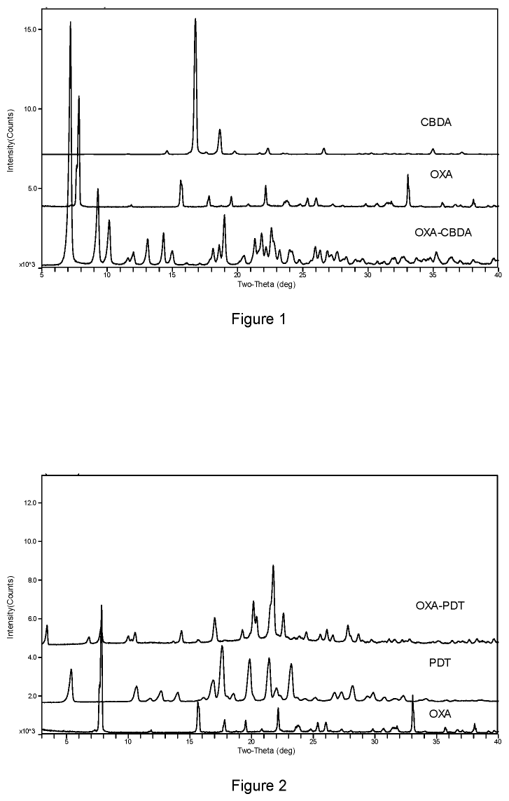

It can be seen from FIG. 2 that oxaliplatin-polydatin cocrystal (OXA-PDT) has characteristic diffraction peaks at 3.4°±0.2°, 6.8°±0.2°, 10°±0.2°, 14.3°±0.2°, 20.4°±0.2°, 22.6°±0.2°, 25.6°±0.2°, and 28.6°±0.2°, which are obviously different from those of raw materials (OXA and PDT). At the same time, the main characteristic peaks of OXA and PDT disappear in cocrystal compound. It shows that the cocrystal compound with new crystal structure is formed by hydrogen bonding between OXA and PDT.

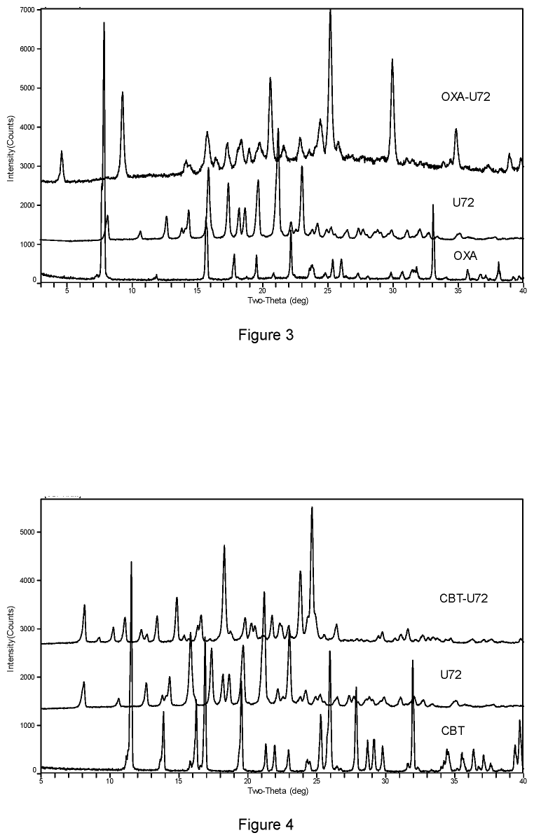

It can be seen from FIG. 3 that the characteristic diffraction peaks of oxaliplatin-chloroquine cocrystal (OXA-U72) are at 4.6°±0.2, 9.3°±0.2, 16.4°±0.2, 19°±0.2°, 20.6°±0.2° and 21.6°±0.2°, which are obviously different from those of raw materials (OXA and U72). At the same time, the main characteristic peaks of OXA and U72 disappear in the cocrystal compounds, indicating that OXA and U72 form a new cocrystal compound by hydrogen bond.

It can be seen from FIG. 4 that carboplatin-chloroquine cocrystal (CBT-U72) has characteristic diffraction peaks at 9.2°±0.2°, 10.2°±0.2°, 11.1°±0.2°, 12.2°±0.2°, 13.4°±0.2°, 14.8°±0.2°, 15.3°±0.2°, 16.6°±0.2°, 20.3°±0.2°, and 20.5°±0.2°, which are obviously different from those of raw materials (CBT and U72). At the same time, the main characteristic peaks of CBT and U72 disappear in cocrystal compound. It shows that the cocrystal compound with new crystal structure is formed between CBT and U72 by hydrogen bonding.

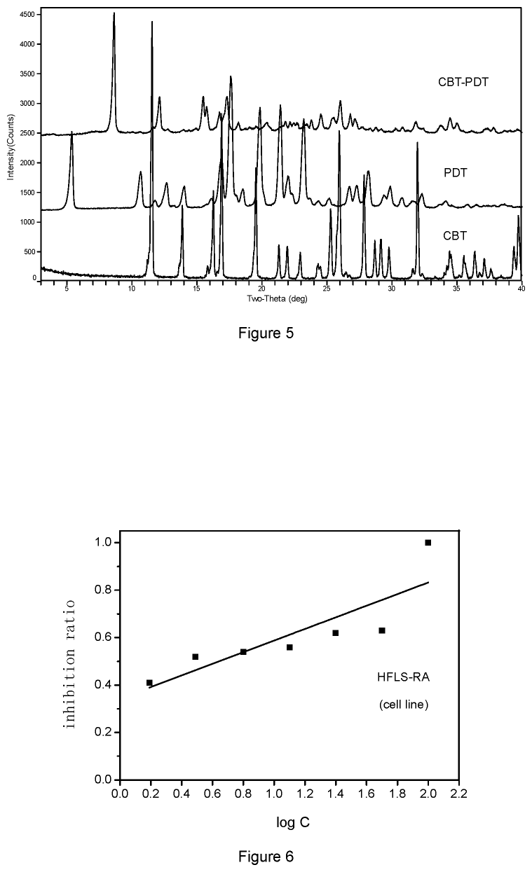

It can be seen from FIG. 5 that carboplatin-polydatin cocrystal (CBT-PDT) has characteristic diffraction peaks at 8.6°±0.2°, 12.1°±0.2°, 15.5°±0.2°, 15.8°±0.2°, 17.3°±0.2°, and 20.3°±0.2°, which are obviously different from those of raw materials (CBT and PDT). At the same time, the main characteristic peaks of CBT and PDT disappear in cocrystal compound, indicating that CBT and PDT form cocrystal compound with new crystal structure by hydrogen bond.

TABLE 1
Test data of equilibrium solubility of platinum-based
drug and platinum-based pharmaceutical cocrystal

| | equilibrium solubility | |
|---|---|---|
| sample | in water (25° C.) | |
| Oxaliplatin (OXA) | 5.0 | mg/mL |
| oxaliplatin-1,1-cyclobutane dicarboxylic acid | 8.05 | mg/mL |
| cocrystal (OXA-CBDA) | | |
| oxaliplatin-polydatin cocrystal (OXA-PDT) | 0.1 | mg/mL |
| oxaliplatin-chloroquine cocrystal (OXA-U72) | 0.4 | mg/mL |
| Carboplatin (CBT) | 12.7 | mg/mL |
| carboplatin-polydatin cocrystal (CBT-PDT) | 16.2 | mg/mL |
| carboplatin-chloroquine cocrystal (CBT-U72) | 0.6 | mg/mL |

The equilibrium solubility of oxaliplatin and carboplatin in oxaliplatin-based cocrystal and carboplatin-based cocrystal was determined by high performance liquid chromatography (HPLC). It can be seen from Table 1 that the equilibrium solubility of oxaliplatin-1,1-cyclobutane dicarboxylic acid cocrystal (OXA-CBDA) and carboplatin-polydatin (CBT-PDT) in aqueous solution is higher than that of oxaliplatin and carboplatin, respectively and they have advantages in solubility.

TABLE 2
Test data of thermal stability at high temperature (60° C.)

| | OXA | OXA-CBDA | OXA-PDT | OXA-U72 | CBT | CBT-PDT | CBT-U72 |
|---|---|---|---|---|---|---|---|
| Sampling | Content of | Content of | Content of | Content of | Content of | Content of | Content of |
| time | OXA/% | OXA/% | OXA/% | OXA/% | CBT/% | CBT/% | CBT/% |
| Day 0 | 100 | 100 | 100 | 100 | 100 | 100 | 100 |
| Day 1 | 98.36 | 100 | 100 | 100 | 99.76 | 100 | 100 |
| Day 3 | 99.33 | 99.98 | 100 | 99.38 | 99.29 | 99.27 | 99.66 |
| Day 5 | 99.18 | 99.12 | 99.73 | 99.46 | 99.16 | 99.08 | 99.84 |
| Day 10 | 99.09 | 99.45 | 99.63 | 99.33 | 99.27 | 99.69 | 99.67 |
| Day 15 | 98.21 | 99.57 | 99.87 | 99.46 | 99.82 | 99.83 | 99.97 |
| crystal | unchanged | unchanged | unchanged | unchanged | unchanged | unchanged | unchanged |
| structure | | | | | | | |

It can be seen from Table 2 that for oxaliplatin-based cocrystal and carboplatin-based cocrystal, the contents of oxaliplatin and carboplatin are basically unchanged after 15 days of continuous high temperature heating test, indicating that the cocrystal compounds have good thermal stability.

TABLE 3
Test data of light stability (Total illumination 1.2 × 10⁶ lux · hr)

| | OXA | OXA-CBDA | OXA-PDT | OXA-U72 | CBT | CBT-PDT | CBT-U72 |
|---|---|---|---|---|---|---|---|
| Sampling | Content of | Content of | Content of | Content of | Content of | Content of | Content of |
| time | OXA/% | OXA/% | OXA/% | OXA/% | CBT/% | CBT/% | CBT/% |
| Day 0 | 100 | 100 | 100 | 100 | 100 | 100 | 100 |
| Day 1 | 98.87 | 100 | 100 | 100 | 99.37 | 100 | 100 |
| Day 3 | 99.32 | 100 | 99.97 | 99.21 | 99.84 | 100 | 99.20 |
| Day 5 | 96.89 | 99.56 | 99.88 | 99.64 | 99.31 | 99.60 | 99.24 |
| Day 10 | 99.45 | 99.97 | 99.44 | 99.33 | 98.29 | 99.72 | 99.57 |
| Day 15 | 98.01 | 99.30 | 99.91 | 99.64 | 98.82 | 99.29 | 99.92 |
| crystal | unchanged | unchanged | unchanged | unchanged | unchanged | unchanged | unchanged |
| structure | | | | | | | |

It can be seen from Table 3 that for oxaliplatin-based cocrystal and carboplatin-based cocrystal, the contents of oxaliplatin and carboplatin are basically unchanged after 15 consecutive days of indoor light test, indicating that their light stability is good.

The effect of oxaliplatin on rheumatoid arthritis can be proved by the results obtained in vivo and in vitro tests.

Human fibroblast-like synovial cell rheumatoid arthritis (HFLS-RA) cells were cultured in vitro and their proliferation was measured by quantifying the number of cells 48 h after treatment. The cells were treated with oxaliplatin at the following concentrations: 1.563 μg/mL, 3.125 μg/mL, 6.25 μg/mL, 12.5 μg/mL, 25 μg/mL, 50 μg/mL, 100 μg/mL. Oxaliplatin significantly decreased the proliferation of HFLS-RA with IC₅₀=4.56 μg/mL. The results are shown in FIG. 6.

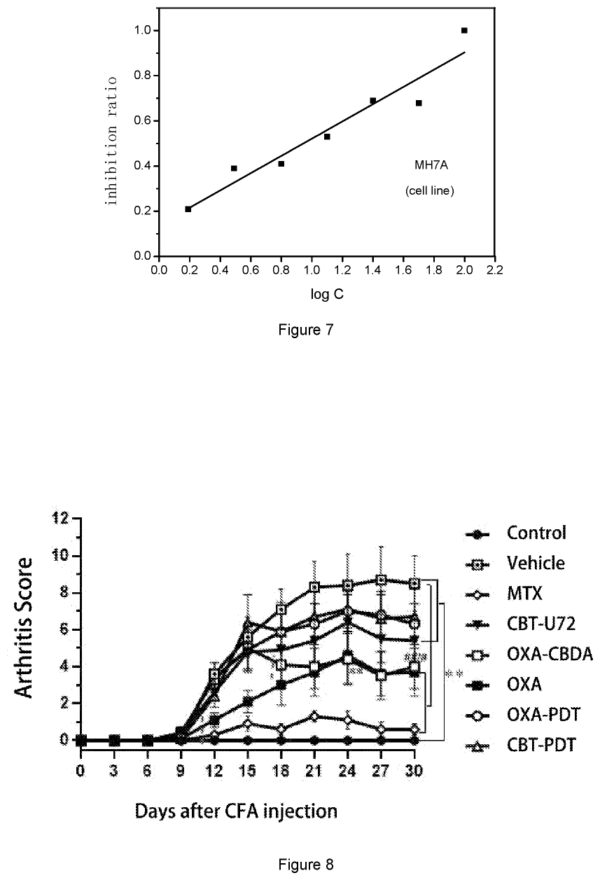

Human MH7A synovial cells were cultured in vitro and measured by quantifying the number of cells 48 h after treatment Proliferation. Cells were treated with oxaliplatin at the following concentrations: 1.563 μg/mL, 3.125 g/mL, 6.25 μg/mL, 12.5 μg/mL, 25 μg/mL, 50 μg/mL, 100 μg/mL. Oxaliplatin significantly reduced the proliferation of MH7A with IC₅₀=8.91 μg/mL. The results are shown in FIG. 7.

As shown in FIG. 6, the proliferation of 99% of HFLS-RA cells was inhibited by oxaliplatin (IC₅₀=4.56 μg/mL); as shown in FIG. 7, the proliferation of 99% of MH7A cells was inhibited by oxaliplatin (IC₅₀=8.91 μg/mL). These results confirmed the antiproliferative efficacy of oxaliplatin in rheumatoid arthritis.

In addition, the effects of platinum-based drug and platinum-based cocrystal on rheumatoid arthritis were studied by using rat adjuvant arthritis (AIA) model. The establishment of AIA was induced by subcutaneous injection of 0.10 mL of heat inactivated Mycobacterium tuberculosis H37Ra suspension (CFA) into the tail root of rats. From the 9th day after AIA modeling, oxaliplatin (OXA), the cocrystal of oxaliplatin and 1,1-cyclobutane dicarboxylic acid (OXA-CBDA), the cocrystal of oxaliplatin and polydatin (OXA-PDT), the cocrystal of carboplatin and chloroquine (CBT-U72), the cocrystal of carboplatin and polydatin (CBT-PDT) were injected intravenously every 3 days. The positive control group (methotrexate, MTX) was given once on day 0, day 3 and day 7 after modeling respectively, and then once a week until the end of the experiment.

Measurement of the posterior toe swelling volume: before modeling, according to the standard operation, the ankle joint of the rat was dotted and permanently marked, and the volume of the left and right hindfoot was measured as the starting point. After modeling, the foot swelling volume was measured every 3 days, and measured three times and the average value was calculated every time, and the joint swelling degree (joint swelling degree=(left and right total foot volume−base point)/2) was recorded and calculated. The differences of swelling degree among groups were compared, and the change curve was drawn.

Limb arthritis index score: from the 9th day after the CFA modeling, the incidence of rats was observed and recorded every day, and the 5-grade scoring method was used for AIA score. Normal condition, there is no redness and swelling, 0 points; mild redness and swelling of local joints or toes, 1 point; moderate redness and swelling of toe joint and plantar joint or ankle joint, 2 points; redness and swelling of all feet below ankle joint or severe swelling of ankle joint, 3 points; redness, swelling and deformation of all feet and claws, 4 points. The cumulative sum of anterior and posterior limb scores was the arthritis score of each rat, and the highest score was 16 points for each rat.

On the 9th day after CFA modeling, inflammation such as redness and swelling began to appear in the hind toes of rats. With the progress of the disease, the forelimbs were gradually involved, and inflammatory nodules appeared in the ear, tail and other parts. Compared with the Vehicle group, the scores of arthritis index in the platinum-based drug and platinum-based cocrystal treatment group were significantly lower than that in the Vehicle group after 12 days (P<0.01 or 0.01 P<0.05), showing a better AIA remission rate. At the same time, compared with the positive control group (MTX), the effect of the therapeutic administration of platinum-based drug and platinum-based cocrystal treatment group (administered from day 9) was similar to that of the preventive administration (from day 0) of positive control group (MTX). The above test results showed that platinum-based drug and platinum-based cocrystal were significantly effective in the treatment of rheumatoid arthritis (see FIG. 8).

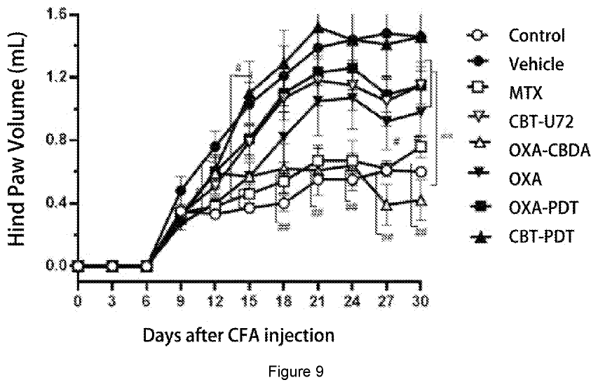

On the 9th day after CFA modeling, inflammation such as redness and swelling began to appear in the hind toes of rats. With the progress of the disease, the forelimbs were gradually involved, and inflammatory nodules appeared in the ear, tail and other parts. Compared with the control group, the swelling degree of the posterior ankle joint in the Vehicle group increased significantly from the 12th day with significance (P<0.01 or 0.01 P<0.05); and compared with the Vehicle group, the swelling degree of in the platinum-based drug and platinum-based cocrystal treatment group was significantly lower than that in the Vehicle group (P<0.01 or 0.01 P<0.05) on the 12th day, 15th day, 27th day and 30th day. At the same time, compared with the positive control group (MTX), the effect of the therapeutic administration of platinum-based drug and platinum-based cocrystal treatment group (administered from day 9) was similar to that of the preventive administration (from day 0) of positive control group (MTX). The above test results showed that platinum-based drug and platinum-based cocrystal were significantly effective in the treatment of rheumatoid arthritis (see FIG. 9).

1. The use of oxaliplatin or a pharmaceutical composition containing oxaliplatin or platinum-based pharmaceutical cocrystal or a pharmaceutical composition containing platinum-based pharmaceutical cocrystal in the preparation of medicaments for the treatment of autoimmune diseases, preferably the autoimmune disease being rheumatoid arthritis, wherein,
the platinum-based pharmaceutical cocrystal is selected from the group consisting of a cocrystal formed by oxaliplatin and 1,1-cyclobutane dicarboxylic acid, a cocrystal formed by oxaliplatin and chloroquine, a cocrystal formed by oxaliplatin and polydatin, a cocrystal formed by carboplatin and chloroquine, and a cocrystal formed by carboplatin and polydatin;
optimally, the cocrystal formed by oxaliplatin and 1,1-cyclobutane dicarboxylic acid contains one or more of the following diffraction peaks in the XRPD profile: 7.2°±0.2°, 9.3°±0.2°, 10.2°±0.2°, 13.1°±0.2°, 14.3°±0.2°, 15°±0.2°, 19°±0.2°, 21.3±0.2°, 21.9°±0.2°, and 22.6°±0.2°, which is basically consistent with FIG. 1; or,
the cocrystal formed by oxaliplatin and polydatin contains one or more of the following diffraction peaks in the XRPD profile: 3.4°±0.2°, 6.8°±0.2°, 10°±0.2°, 14.3°±0.2°, 20.4°±0.2°, 22.6°±0.2°, 25.6°±0.2°, and 28.6°±0.2°, which is basically consistent with FIG. 2; or,
the cocrystal formed by oxaliplatin and chloroquine contains one or more of the following diffraction peaks in the XRPD spectrum: 4.6°±0.2°, 9.3°±0.2°, 16.4°±0.2°, 19°±0.2°, 20.6°±0.2°, and 21.6°±0.2°, which is basically consistent with FIG. 3; or,
the cocrystal formed by carboplatin and chloroquine contains one or more of the following diffraction peaks in the XRPD profile: 9.2°±0.2°, 10.2°±0.2°, 11.1°±0.2°, 12.2°±0.2°, 13.4°±0.2°, 14.8°±0.2°, 15.3°±0.2°, 16.6°±0.2°, 20.3°±0.2°, and 20.5°±0.2°, which is basically consistent with FIG. 4; or,
the cocrystal formed by carboplatin and polydatin contains one or more of the following diffraction peaks in the XRPD spectrum: 8.6°±0.2°, 12.1°±0.2°, 15.5°±0.2°, 15.8°±0.2°, 17.3°±0.2°, and 20.3°±0.2°, which is basically consistent with FIG. 5; or
in the pharmaceutical composition containing oxaliplatin or the pharmaceutical composition containing platinum-based pharmaceutical cocrystal, oxaliplatin or one or more of the platinum-based pharmaceutical cocrystal is the principal or only active substance of the pharmaceutical composition;
optionally, the pharmaceutical composition may further contain at least one therapeutic agent or one adjuvant therapeutic agent; preferably, at least one therapeutic agent or an adjuvant therapeutic agent is selected from the group consisting of chloroquine, hydroxychloroquine, methotrexate, curcumin, polydatin, glutathione, and aloe vera;
optionally, the pharmaceutical composition further contains a pharmaceutically acceptable carrier or excipient;
optionally, the pharmaceutical composition may be administered by oral, buccal mucosa, inhalation spray, sublingual, transdermal, transmucosal, topical, muscular, subcutaneous, intradermal, or intravenous route.

2. The platinum-based pharmaceutical cocrystal, which is the cocrystal formed by oxaliplatin and 1,1-cyclobutane dicarboxylic acid, the cocrystal formed by oxaliplatin and chloroquine, the cocrystal formed by oxaliplatin and polydatin, the cocrystal formed by carboplatin and chloroquine, or the cocrystal formed by carboplatin and polydatin.

3. The cocrystal according to claim 2, which is the cocrystal formed by oxaliplatin and 1,1-cyclobutane dicarboxylic acid, preferably the cocrystal formed by oxaliplatin and 1,1-cyclobutane dicarboxylic acid contains one or more of the following diffraction peaks in the XRPD spectrum: 7.2°±0.2°, 9.3°±0.2°, 10.2°±0.2°, 13.1°±0.2°, 14.3°±0.2°, 15°±0.2°, 19°±0.2°, 21.3°±0.2°, 21.9°±0.2°, and 22.6°±0.2°, which is basically consistent with FIG. 1.

4. The cocrystal according to claim 2, which is the cocrystal formed by oxaliplatin and polydatin, preferably, the cocrystal formed by oxaliplatin and polydatin contains one or more of the following diffraction peaks in the XRPD pattern: 3.4±0.2°, 6.8°±0.2°, 10°±0.2°, 14.3°±0.2°, 20.4°±0.2°, 22.6°±0.2°, 25.6°±0.2°, and 28.6°±0.2°, which is basically consistent with FIG. 2.

5. The cocrystal according to claim 2, which is the cocrystal formed by oxaliplatin and chloroquine, preferably, the cocrystal formed by oxaliplatin and chloroquine contains one or more of the following diffraction peaks in the XRPD pattern: 4.6°±0.2°, 9.3°±0.2°, 16.4°±0.2°, 19°±0.2°, 20.6°±0.2°, and 21.6°±0.2°, which is basically consistent with FIG. 3.

6. The cocrystal according to claim 2, which is the cocrystal formed by carboplatin and chloroquine, preferably, the cocrystal formed by carboplatin and chloroquine contains one or more of the following diffraction peaks in the XRPD pattern: 9.2°±0.2°, 10.2°±0.2°, 11.1°±0.2°, 12.2°±0.2°, 13.4°±0.2°, 14.8°±0.2°, 15.3°±0.2°, 16.6°±0.2°, 20.3°±0.2°, and 20.5°±0.2°, which is basically consistent with FIG. 4.

7. The cocrystal according to claim 2, which is the cocrystal formed by carboplatin and polydatin, preferably, the cocrystal formed by carboplatin and polydatin contains one or more of the following diffraction peaks in the XRPD pattern: 8.6°±0.2°, 12.1°±0.2°, 15.5°±0.2°, 15.8°±0.2°, 17.3°±0.2°, and 20.3°±0.2°, which is basically consistent with FIG. 5.

8. A pharmaceutical composition, which contains platinum-based pharmaceutical cocrystal, wherein the platinum-based pharmaceutical cocrystal is selected from oxaliplatin cocrystal or carboplatin cocrystal, wherein oxaliplatin cocrystal is selected from the group consisting of a cocrystal formed by oxaliplatin and 1,1-cyclobutane dicarboxylic acid, a cocrystal formed by oxaliplatin and chloroquine, and a cocrystal formed by oxaliplatin and polydatin; carboplatin cocrystal is selected from the group consisting of a cocrystal formed by carboplatin and chloroquine, and a cocrystal formed by carboplatin and polydatin.

9. The pharmaceutical composition according to claim 8, wherein the platinum-based pharmaceutical cocrystal is defined in any of claims 2-7.

10. The pharmaceutical composition according to any of claim 8-9, wherein one or more of oxaliplatin cocrystal and carboplatin cocrystal is the main or only active substance of the pharmaceutical composition.

11. The pharmaceutical composition according to any of claim 8-10, which may further contain at least one therapeutic agent or one adjuvant.

12. The pharmaceutical composition according to claim 11, wherein at least one therapeutic agent or adjuvant is selected from the group consisting of chloroquine, hydroxychloroquine, methotrexate, curcumin, polydatin, glutathione and aloe vera.

13. The pharmaceutical composition according to any of claim 8-12, which further contains a pharmaceutically acceptable carrier or excipient.

14. The pharmaceutical composition according to any of claim 8-13, wherein the pharmaceutical composition may be administered by oral, buccal mucosa, inhalation spray, sublingual, transdermal, transmucosal, local, muscular, subcutaneous, intradermal, or intravenous route.

15. A method of treating disease in a subject in need, which includes administering to the subject the cocrystal or oxaliplatin according to any of claims 2-7 or the pharmaceutical composition according to any of claims 8-14 or the pharmaceutical composition containing oxaliplatin, wherein the cocrystal or oxaliplatin or the pharmaceutical composition has a therapeutically effective dose.

16. The method according to claim 15, wherein the disease is an autoimmune disease.

17. The method according to claim 16, wherein the autoimmune disease is rheumatoid arthritis.

18. The method according to any of claim 15-17, wherein the therapeutically effective dose of the cocrystal or oxaliplatin or the pharmaceutical composition is about 0.01 to about 10 mg/kg body weight.

19. The method according to any of claim 15-18, wherein the therapeutically effective dose of the cocrystal or oxaliplatin or the pharmaceutical composition is about 0.01 to about 5 mg/kg body weight.

20. The method according to any of claim 15-19, the pharmaceutical composition is an aqueous composition or a suspension composition containing at least one therapeutic agent or adjuvant that is dissolved or dispersed in a pharmaceutically acceptable amount.
